(19) United States
(12) Patent Application Publication
(10) Pub. No.: US 2022/0028504 A1
(43) Pub. Date: Jan. 27, 2022

(54) SECURE, INTEGRATED, PERSONALIZED SMART INFORMATION MANAGEMENT AND EXCHANGE SYSTEMS, METHODS, AND DEVICES

(71) Applicants: Qi Luo, Plano, TX (US); Joshua J Luo, Plano, TX (US); Jessie Luo, Plano, TX (US)

(72) Inventors: Qi Luo, Plano, TX (US); Joshua J Luo, Plano, TX (US); Jessie Luo, Plano, TX (US)

(73) Assignees: Qi Luo, Plano, TX (US); Joshua J Luo, Plano, TX (US); Jessie Luo, Plano, TX (US)

(21) Appl. No.: 16/939,144

(22) Filed: Jul. 27, 2020

(51) Int. Cl. G16H 10/60; G16H 40/67; G06K 7/14; G06F 21/62; G06N 5/02; A61B 5/0205

(52) U.S. Cl. CPC G16H 10/60; G16H 40/67; G06K 7/1413; G06Q 50/265; G06F 21/6218; G06N 5/022; A61B 5/02055; G06K 7/1417

(57) ABSTRACT

Secure, integrated, personalized smart information management and exchange systems, methods and devices are disclosed to increase the quality of professional services and reduce the cost of operations. The present invention is smart by providing cognitive augmented interaction based on dynamically updated guidelines, knowledge bases, and/or artificial intelligences. The present invention also is secure for both stored and transmitted information. The present invention focuses on the individual level to provide personalized and data driven feedbacks. The present invention integrates various fields of inputs to provide whole and more accurate services.

### CROSS-REFERENCE TO RELATED APPLICATIONS

Not applicable

### STATEMENT REGARDING FEDERALLY SPONSORED RESEARCH OR DEVELOPMENT

Not applicable

### STATEMENT REGARDING SEQUENCE LISTING OR PROGRAM

Not applicable

### FIELD OF TECHNOLOGY

The present invention relates generally to information management and exchange systems, methods, and devices, and more particularly, to professional service sectors which offer customized, knowledge-based information and consulting.

### BACKGROUND

As the explosion of information and advancement in technology, more and more people are rely upon information management and exchange systems to help their daily life, especially in the fields that need professional services that provide customized, knowledge-based information and consulting. Those professional services include, but not limited to, healthcare, law, finance, etc.

The information related to above mentioned areas are usually sensitive and regulated. Although there are laws, for example, The Health Insurance Portability and Accountability Act (HIPPA), to guard the information's privacy and confidentiality, most of those systems currently are focused on security in the system level, not at the individual level, and there were quite a few incidences for data breaches and modification data attacks.

Currently most of those information management and exchange systems are proprietary. It is very difficult for them to talk to each other smoothly because they use their own proprietary concept coding systems; for them to communicate each other without misunderstanding, a new interface and/or a crosswalk system are needed to be developed. Besides, sometimes even the most complicated systems, e.g., electronic medical record systems, are missing the capabilities to transmit some of very important information, which increase the cost of operations.

Currently most of those information management and exchange systems are designed to be static and one-size-fits-all. Their outputs are not dynamic nor personalized based on input data. For example, when searching with keyword “headache”, the revealed search results will be exact the same regardless of searchers' gender or searchers' age.

Currently most of those information management and exchange systems are served as passive record keeping systems. They are not capable of providing cognitive augmented interaction based on guidelines, knowledge bases, and/or artificial intelligences.

The present invention is disclosed to resolve the above mentioned issues.

### SUMMARY OF THE INVENTION

The purpose of present invention is to provide secure, integrated, personalized smart information management and exchange systems, methods, and devices so that people can make data-driven decisions based on feedback information provided by the systems. While the systems are applicable in various professional fields, we will focus on the health care sector to illustrate exemplary embodiments of the present invention.

### BRIEF DESCRIPTION OF THE DRAWINGS

The foregoing and other features and advantages of the present invention will be apparent from the following, more particular description of exemplary embodiments of the invention, as illustrated in the accompanying drawings wherein like reference numbers generally indicate identical, functionally similar, or structurally similar elements. The left most digits in the corresponding reference number indicate the drawing in which an element first appears.

FIG. 1 depicts an overview diagram of an exemplary embodiment of secure, integrated, personalized smart information management and exchange systems according to the present invention;

FIG. 2 depicts features of an exemplary embodiment for a public permission network

FIG. 3 depicts methods of an exemplary embodiment to assign a unique identifier

FIG. 4 depicts methods of an exemplary embodiment to attach time stamps and geolocation coordinates

FIG. 5 depicts methods of an exemplary embodiment to extract, standardize, and incorporate related data

FIG. 6 depicts methods of an exemplary embodiment to secure data

FIG. 7 depicts methods of an exemplary embodiment to map and standardize the concept codes

FIG. 8 depicts methods of an exemplary embodiment to interpret different languages and different sentiments

FIG. 9 depicts methods of an exemplary embodiment to provide data driven actionable insights

FIG. 10 depicts exemplary devices

### DETAILED DESCRIPTION OF EMBODIMENTS OF THE PRESENT INVENTION

The following detailed description for one of the preferred embodiments presents a description of certain specific embodiments to assist in understanding the claims. However, the present invention can be embodied in a multitude of different ways as defined and covered by the claims.

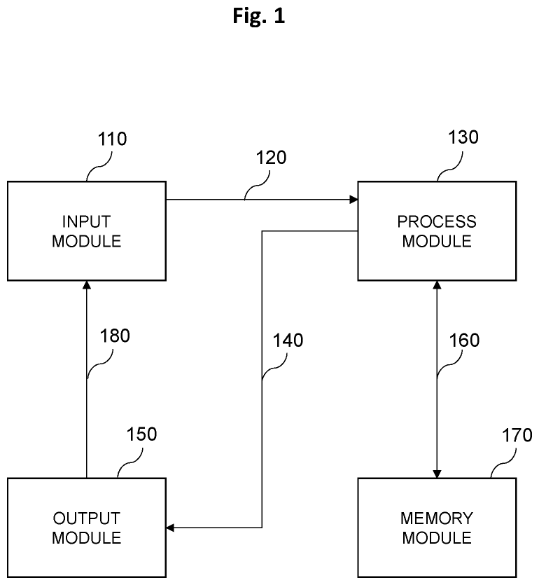

Referring to FIG. 1, the components for one of the preferred embodiments of secure, integrated, personalized smart information management and exchange systems of the present invention are shown.

The systems are comprising of an input module 110, a process module 130, a memory module 150, and an output module 170.

The input module is configured for a user to input data into the systems. It includes at least one of the followings:
i. a keyboard to type in alphanumerical symbols
ii. a computer mouse to control cursor positions
iii. a microphone or a set of microphones to capture and digitalize sounds
iv. a scanner, including bar/QR code scanners
v. a digitizer pen
vi. a digital camera or a set of cameras, including video cameras
vii. a touch screen
viii. a sensor or a set of sensors for global position, body temperature, blood sugar concentration, blood pressure, pulse rate, oxygen saturation, ultrasound, muscle contraction, eye movement, electromyography, electrocardiogram, electroencephalogram, air temperature, air pressure, air humidity, sea level, compass
ix. a digitizer clock, including time signals
x. any software virtual versions for the above mentioned devices
xi. any combinations or permutations of above mentioned devices

The output module is configured for a user to receive information from the systems. It includes at least one of the followings:
i. a displayer or a set of displayers
ii. a projector or a set of projectors
iii. a speaker or a set of speakers
iv. a printer or a set of printers, including 3D printers
v. a plotter
vi. any software virtual versions for the above mentioned devices
vii. any combinations or permutations of above mentioned devices

The memory module is collectively configured to store software programs and the input/output data and some temporary transmitted data sets in between.

The process module is configured to process the data by carrying out the software program instructions and convert the input data into actionable insights.

The connection 120 is configured to push the data from the input module to the process module. The connection 140 is configured for the output module to receive actionable insights from the process module. The connection 160 is configured for two way communication between the process module and the memory module. The connection 180 is configured for the input module to get information from the output module.

The functions of those connections are realized by at least one of the followings:
i. a computer bus or a set of computer buses
ii. a wire or a set of wires
iii. a cable or a set of cables, including optical fibers
iv. a set frequency of wireless waves, including, but not limited to:
   a. television and radio wave
   b. satellite communication
   c. mobile telephone communication systems, including 5G
   d. global positioning system or any other equalities
   e. bluetooth
   f. paging
   g. local area networks, including wireless
   h. infrared communication
v. an internet/intranet communication
vi. a communication between/among clouds
vii. any combinations and permutations of above mentioned means

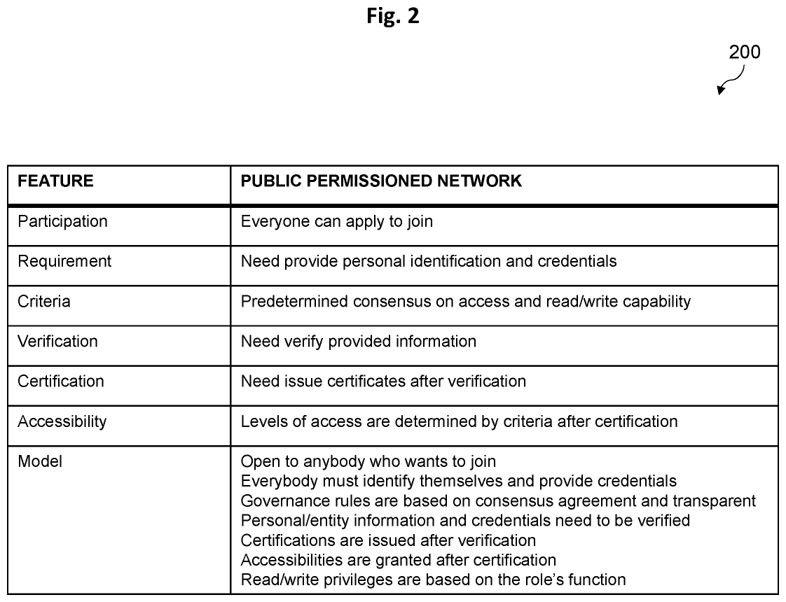

FIG. 2 200 illustrates the features for the public permissioned network that the secure, integrated, personal smart information management and exchange systems of the present invention resided in.

The public permissioned network is open to anyone who wants to join. But everybody must identify themselves, present their credentials, and get the permission before they can join. The governance rules are predetermined by consensus agreement and are transparent. Personal/entity information and credentials need to be verified. Certifications are issued after verification. Accessibilities are granted after certification. Read/write privileges are based on the personal/entity's function.

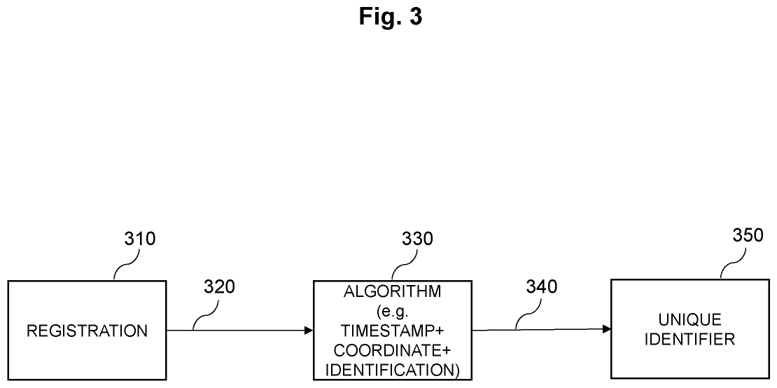

FIG. 3 illustrates the methods to assign a unique identifier to each registrant.

During registration 310, an algorithm 330 is performed to generate a unique identifier 350 for each registrant. The algorithm is to incorporate time stamps, geolocation coordinates, and other personal identification information.

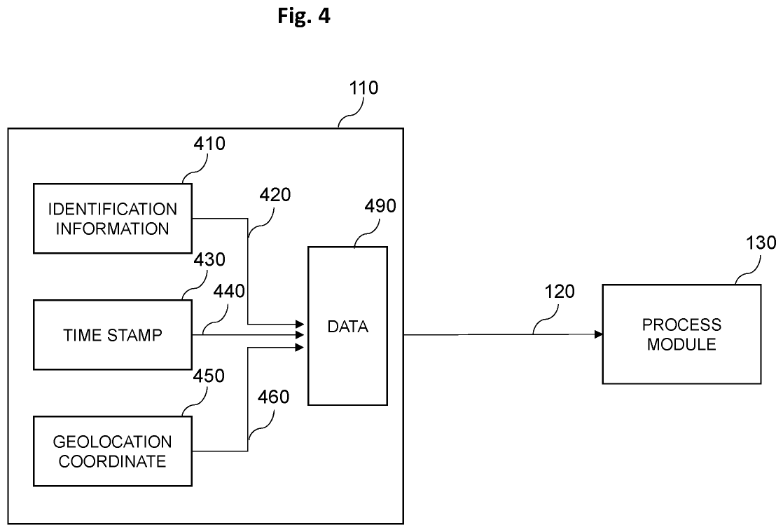

FIG. 4 illustrate the methods to attach time stamps 430, geolocation coordinates 450, and other identification information 410 with other input data 490 inside the input module 110.

The identification information, along with the time stamps and geolocation coordinates if available, is attached to other input data by the instructions of the program.

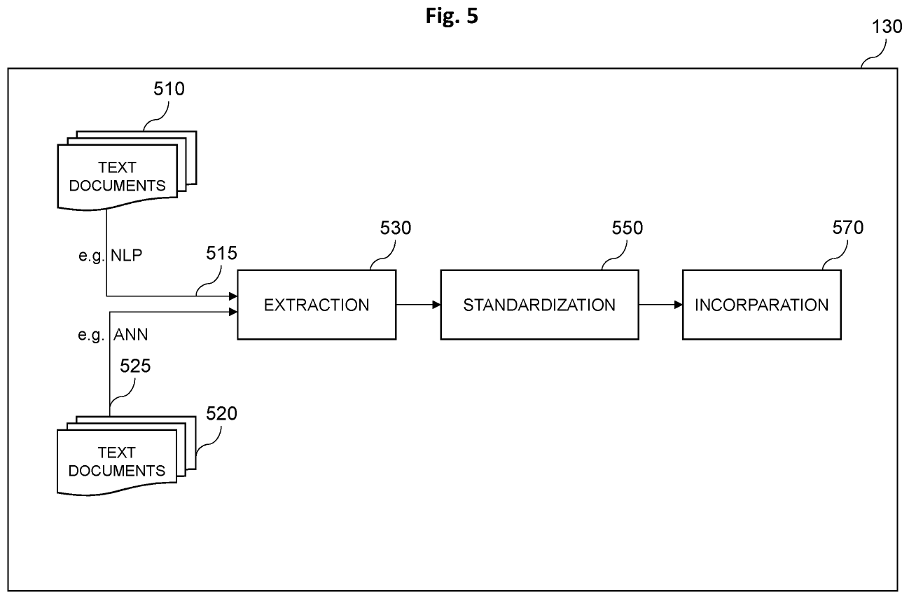

FIG. 5 depicts methods to extract 530, standardize 550, and incorporate 570 related data, which have two basic types: text and image.

Texts documents 510 are parsed through some programs 515, e.g. the natural language processing, then information is extracted, standardized, and incorporated into other related data. While image documents 520 are analyzed by some programs 525, e.g. the artificial neural network, then information is extracted, standardized, and incorporated into other related data.

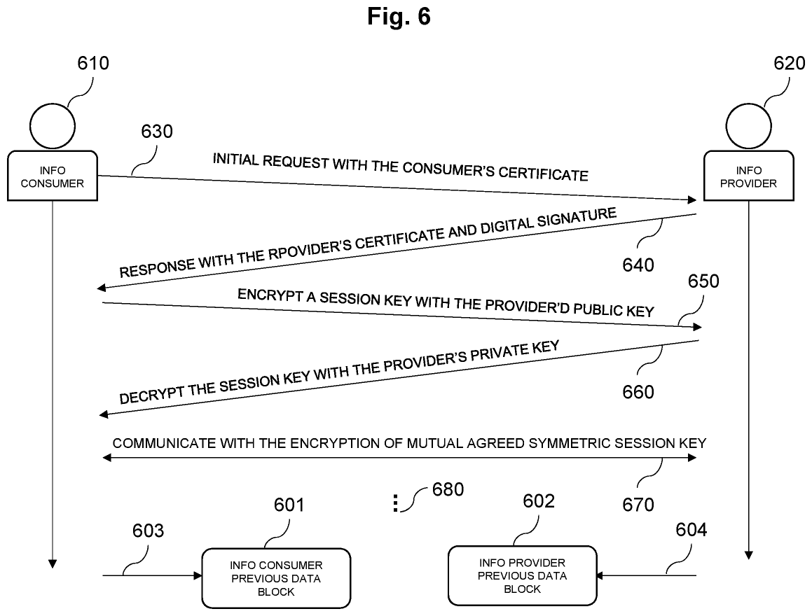

FIG. 6 depicts methods to secure data. The followings are detailed sequential steps for two parties, one is an information consumer 610 and the other is an information provider 620, to communicate securely:
1. When a session starts, an information consumer initiates a request to an information provider of choice with the information consumer's certificate including the consumer's public key, 630;
2. After verification, the information provider of choice responses with the provider's digital signature and certificate including the provider's public key, 640;
3. After verification, the consumer encrypts a session key by using the public key, 650;
4. After receiving, the provider decrypts the session key with the provider's private key, 660;
5. After verification, both parties communicate with the encryption of the mutually agreed symmetrical session key, 670;
6. The communication continues until the end of the session, 680;
7. Upon predetermined rules, the contents of the session's communication are added to the consumer's previous data block 601, 603; the contents of the session's communication are also added to the provider's previous data block 602, 604; those added data blocks are chained according chronological order and are immutable after verification.

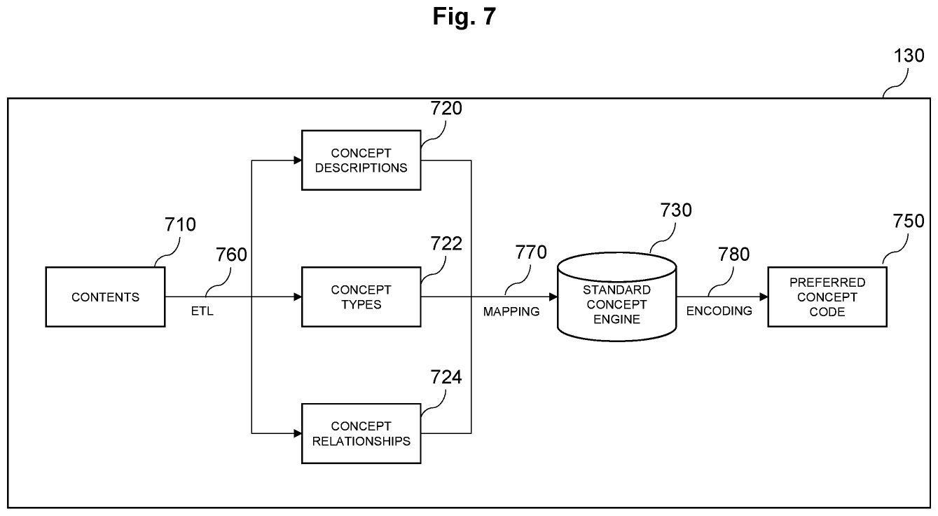

FIG. 7 depicts methods to map and standardize the concept codes.

The contents 710 from various sources are extracted, transformed, and loaded 760 into at least three components:
1. Concept description 720
2. Concept types 722
3. Concept relationships 724

Those components are mapped with 770 the concepts in the standard concept engine 730. Then the mapped concepts are encoded 780 to the preferred concept codes 750 for later use. If no exact matches are found, acceptable concepts or synonyms are selected.

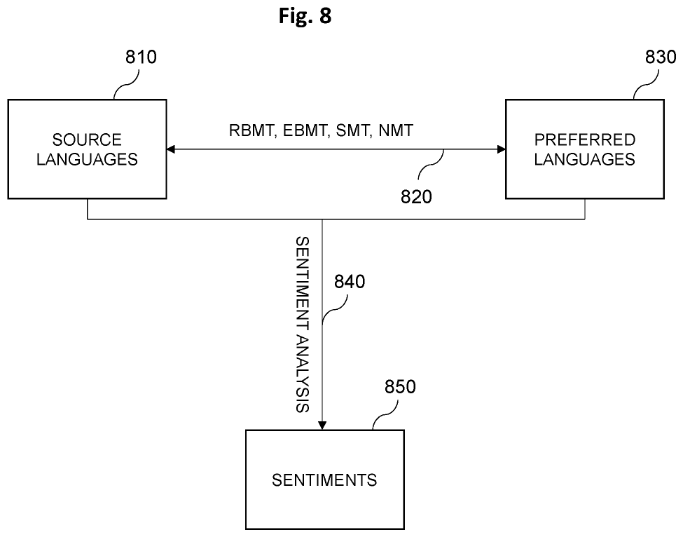

FIG. 8 depicts methods to interpret different languages and different sentiments.

The contents in the source languages 810 can be translated 820 to the contents in the preferred language 830, and vice versa, by using methods such as rule-based machine translation, example-based machine translation, statistical machine translation, and neural machine translation.

The sentiments 850 can be extracted 840 from the contents both in source languages and in preferred languages by using methods such as knowledge-based techniques, statistical methods, and hybrid approaches.

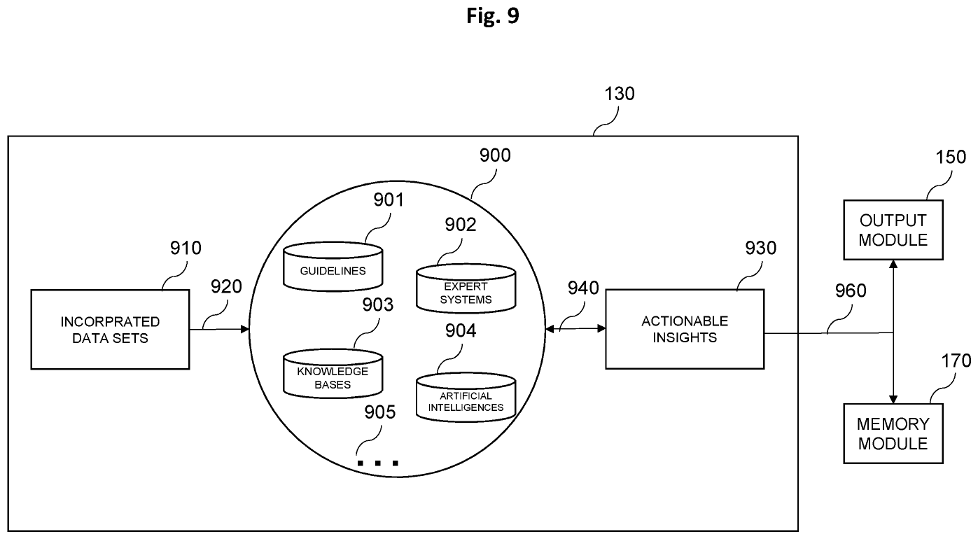

FIG. 9 depicts methods to provide data driven actionable insights.

The incorporated data sets 910 are processed by mining, comparing, and matching with various engines 900, including guidelines 901, expert systems 902, knowledge bases 903, artificial intelligences 904, and others 905. The actionable insights 930 are generated from those processes, and either being presented to the output module 150 or being sent to the memory module 170 for storage. Those newly generated actionable insights, with or without other new information, can be sent back and incorporated with those engines to update them.

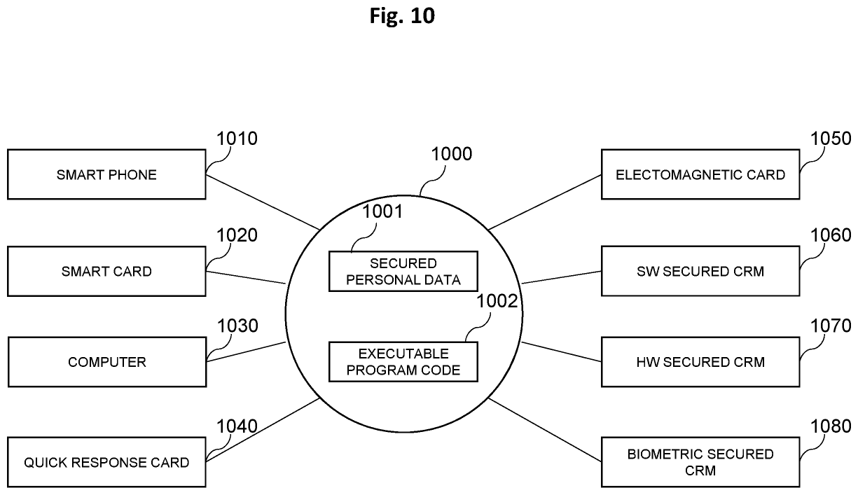

FIG. 10 depicts exemplary devices.

Those devices 1000 store executable program instruction codes 1002 and personal data 1001 securely, can be read, written, and executed by machine locally or remotely, and perform the function of information management and exchange. The followings are some of the examples:
1. a smart phone 1010
2. a smart card 1020
3. a computer, including desktop, laptop, tablet, and hand-held 1030
4. a machine readable printed quick response card 1040
5. a machine readable electromagnetic card 1050
6. a software secured computer readable medium, including hard disk, laser disk, memory cards of various sizes and formats 1060
7. a hardware secured computer readable medium 1070
8. a biometric secured computer readable medium 1080

What is claimed is:

1. Smart systems for providing secure, integrated, personalized information management and exchange, the smart systems comprising:
a. an input module configured for a person to input data
I. the input module further comprising at least one of a plurality of following input devices:
i. a keyboard to type in alphanumerical symbols
ii. a computer mouse
iii. a microphone or a set of microphones to capture and digitalize sounds
iv. a scanner, including bar/QR code scanners
v. a digitizer pen
vi. a digital camera or a set of cameras, including video cameras
vii. a touch screen
viii. a sensor or a set of sensors for global position, body temperature, blood sugar concentration, blood pressure, pulse rate, oxygen saturation, ultrasound, muscle contraction, eye movement, electromyography, electrocardiogram, electroencephalogram, air temperature, air pressure, air humidity, sea level, compass
ix. a digitizer clock, including time signals
x. any software virtual versions for the above mentioned devices
xi. any combinations or permutations of above mentioned devices
b. an output module configured to present the feedbacks from the systems
I. the output module further comprising at least one of a plurality of following output devices:
i. a displayer or a set of displayers
ii. a projector or a set of projectors
iii. a speaker or a set of speakers
iv. a printer or a set of printers, including 3D printers
v. a plotter
vi. any software virtual versions for the above mentioned devices
vii. any combinations or permutations of above mentioned devices
c. a memory module configured to store software program instructions and the input/output data and some temporary transmitted data sets in between
d. a processing module configured to process the data by carrying out instructions of software programs to convert input data to outputted actionable insights
e. a means to provide paths for data transmission among those modules and between the sender(s) and the receiver(s) via networks, including, but not limited to:
I. a computer bus or a set of computer buses
II. a wire or a set of wires
III. a cable or a set of cables, including optical fibers
IV. a set frequency of wireless waves, including, but not limited to:
i. television and radio wave
ii. satellite communication
iii. mobile telephone communication systems, including 5G
iv. global positioning system or any other equalities
v. bluetooth
vi. paging
vii. local area networks, including wireless
viii. infrared communication
V. an internet/intranet communication
VI. a communication between/among clouds
VII. any combinations and permutations of above mentioned means.

2. The smart system of claim 1 is a public permissioned network.

3. The smart system of claim 1 has the means to uniquely identify the person.

4. The smart system of claim 1 has the means to record time stamps and geolocation coordinates, if available.

5. The smart system of claim 1 has the means to extract, standardize, and incorporate related data from other systems.

6. The smart system of claim 1 has the means to secure the data/information.

7. The smart system of claim 1 has the means to map and standardize the concept codes.

8. The smart system of claim 1 has the means to interpret different languages and different sentiments.

9. The smart system of claim 1 has the means to provide data driven actionable insights.

10. The smart system of claim 1 has various ways to transmit data/information among those modules.

11. Methods for smart information management and exchange, the methods comprising:
receiving, with or without feedbacks from results of the systems' output, an input from a user
determining the next steps based on at least one of the pluralities of guidelines, knowledge bases, expert systems, and/or artificial intelligences storing all the related data in the memory module.

12. The methods for smart information management and exchange of claim 11, forming a public permissioned network.

13. The methods for smart information management and exchange of claim 11, assigning each person a unique identifier.

14. The methods for smart information management and exchange of claim 11, attaching each record a time stamp and a geolocation coordinate if available.

15. The methods for smart information management and exchange of claim 11, extracting, standardizing, and incorporating related data from other systems.

16. The methods for smart information management and exchange of claim 11, securing related data/information for storage and transmission.

17. The methods for smart information management and exchange of claim 11, mapping and standardizing the concept codes.

18. The methods for smart information management and exchange of claim 11, interpreting different languages and different sentiments.

19. The methods for smart information management and exchange of claim 11, providing data driven advice based on various resources, including but not limited to: guidelines, knowledge bases, expert systems, and artificial intelligences.

20. Devices storing secured personal data and executable program instruction codes, being readable, writable and executable by machines locally or remotely, and performing the function of information management and exchange, comprising at least one of following:
I. a smart phone
II. a smart card
III. a computer, including desktop, laptop, tablet, and hand-held
IV. a machine readable printed quick response card
V. a machine readable electromagnetic card
VI. a software secured computer readable medium, including hard disk, laser disk, memory cards of various sizes and formats
VII. a hardware secured computer readable medium
VIII. a biometric secured computer readable medium.
